(19) United States
(12) Patent Application Publication
(10) Pub. No.: US 2014/0275559 A1
(43) Pub. Date: Sep. 18, 2014

(54) PREPARATION OF CERTAIN (SUBSTITUTED-PHENYL)-TRIAZOLYL-(SUBSTITUTED PHENYL) MOLECULES, AND INTERMEDIATES AND INSECTICIDES RELATED THERETO

(71) Applicant: Dow AgroSciences LLC, Indianapolis, IN (US)

(72) Inventors: James M. RENGA, Indianapolis, IN (US); Anne M. WILSON, Indianapolis, IN (US); Peter BORROMEO, Fishers, IN (US); Carl DeAMICIS, Indianapolis, IN (US); Jerod PATZNER, Indianapolis, IN (US)

(73) Assignee: Dow AgroSciences LLC, Indianapolis, IN (US)

(21) Appl. No.: 14/192,429

(22) Filed: Feb. 27, 2014

Related U.S. Application Data
(60) Provisional application No. 61/778,516, filed on Mar. 13, 2013.

(51) Int. Cl. C07D 249/08; C07C 251/24

(52) U.S. Cl. CPC C07D 249/08; C07C 251/24
USPC 548/265.8; 560/34; 562/439; 564/226

(57) ABSTRACT

This document is related to the preparation of certain (substituted-phenyl)-triazolyl-(substituted phenyl) molecules, and intermediates related thereto, where said intermediates are useful in preparing certain insecticides.

### CROSS REFERENCE TO RELATED APPLICATIONS

This application claims the benefit of U.S. Provisional Patent Application Ser. No. 61/778,516 filed Mar. 13, 2013, the entire disclosure of which is hereby expressly incorporated by reference.

### FIELD OF THE DISCLOSURE

This document is related to the preparation of certain (substituted-phenyl)-triazolyl-(substituted phenyl) molecules, and intermediates related thereto, where said intermediates are useful in preparing certain insecticides.

### BACKGROUND OF THE DISCLOSURE

U.S. Pat. No. 8,178,658 discloses pesticidal compositions comprising a compound having the following structure:

[chemical structure]

wherein Ar₁, Het, Ar₂, J, L, K, Q, R1, R2, R3, and R4 are disclosed in the patent. While processes are disclosed on how to make such compounds, and such processes are useful, it is desired to have more useful processes to make these compounds. In particular, it is desirable to have more useful routes to certain substituted triaryl intermediates disclosed in the patent that are useful in producing the compounds of Formula A-1.

### DETAILED DESCRIPTION OF THE DISCLOSURE

Throughout this document, all temperatures are given in degrees Celsius, and all percentages are weight percentages unless otherwise stated.

The term “alkyl”, as well as derivative terms such as “haloalkyl” and “haloalkoxy”, as used herein, include within their scope straight chain, branched chain and cyclic moieties. Thus, typical alkyl groups are methyl, ethyl, propyl, butyl, pentyl, hexyl, 1-methylethyl, 1,1-dimethylethyl, 1-methylpropyl, 2-methylpropyl, cyclopropyl, cyclobutyl, cyclopentyl and cyclohexyl. The term “alkenyl”, as used herein, means an acyclic, unsaturated (at least one carbon-carbon double bond), branched or unbranched substituent consisting of carbon and hydrogen, for example, vinyl, allyl, butenyl, pentenyl or hexenyl. The term “alkynyl”, as used herein, means an acyclic, unsaturated (at least one carbon-carbon triple bond), branched or unbranched substituent consisting of carbon and hydrogen, for example, ethynyl, propargyl, butynyl, pentynyl or hexynyl. The terms “haloalkyl” and “haloalkoxy” includes alkyl or alkoxy groups substituted with from one to the maximum possible number of halogen atoms, all combinations of halogens included. The term “halogen” or “halo” includes fluorine, chlorine, bromine and iodine, with fluorine being preferred.

[chemical structure]

Haloalkoxyarylhydrazines of Formula 1.2, wherein R is a (C₁-C₆)haloalkoxy, such as, but not limited to, trifluoromethoxy and pentafluoroethoxy, can be prepared as illustrated in Scheme 1. In general, in step a, a haloalkoxyaniline of Formula 1 is reacted with sodium nitrite (NaNO₂) to produce an intermediate diazonium salt of Formula 1.1. In step b, the intermediate diazonium salt is reduced to form a haloalkoxyarylhydrazine of Formula 1.2.

In Step a, approximately a 1:1 molar ratio of the haloalkoxyaniline and NaNO₂ may be used, however, molar ratios of about 1:2 may also be used. This reaction is conducted in a polar protic solvent. Suitable examples of polar protic solvents are water, formic acid, n-butanol, isopropanol ethanol (EtOH), methanol (MeOH), acetic acid (AcOH), and mixtures thereof. Currently, it is preferred if water is used. Furthermore, Step a is conducted in the presence of an inorganic acid. Suitable examples are hydrochloric acid (HCl), nitric acid (HNO₃), phosphoric acid (H₃PO₄), sulphuric acid (H₂SO₄), boric acid (H₃BO₃), hydrofluoric acid (HF), hydrobromic acid (HBr), perchloric acid (HClO₄), tetrafluoroboric acid (HBF₄), and mixtures thereof. Currently, it is preferred if HCl is used. The pH of the reaction is from about −1 to about 4, preferably from about −1 to about 1. The reaction is conducted at a temperature from about −10° C. to about 5° C. and preferably from about −5° C. to about 5° C. The reaction is conducted at about atmospheric pressure, however, higher or lower pressures can be used.

Step b is conducted in a polar, protic solvent. Suitable examples of polar, protic solvents are water, formic acid, n-butanol, isopropanol, EtOH, MeOH, AcOH, and mixtures thereof. Currently, it is preferred if water is used. This reaction is conducted in the presence of a reducing agent, such as, for example, sodium dithionite (Na₂S₂O₄), tin (II) chloride (SnCl₂), hydrogen, and ammonium formate. The pH of the reduction reaction mixture is from about 8 to about 14, and preferably from about 9 to about 12. The reaction is conducted at a temperature from about −10° C. to about 10° C. preferably about −5° C. to about 5° C. The reaction is conducted at about atmospheric pressure, however, higher or lower pressures can be used.

If desired, the haloalkoxyarylhydrazines can be obtained as a salt or a free base ((HX)ₙ where n=0, 1, or 2) with pH adjustments following the reaction in step b.

[chemical structure]

Arylalkoxyimidate salts of Formula 2.2, wherein R₁ is NO₂, C(═O)OH or a (C₁-C₆) ester thereof (C(═O)O(C₁-C₆)alkyl), for example, methyl (C(═O)OCH₃) or ethyl ester (C(═O)OCH₂CH₃), can be prepared as outlined in Scheme 2. In step a, benzonitriles of Formula 2.1 are reacted with anhydrous inorganic acids in an alcohol to produce said arylalkoxyimidate salts, wherein R₂ is (C₁-C₆)alkyl.

In Step a benzonitriles of Formula 2.1 are treated with an anhydrous inorganic acid (HX, wherein X is F, Cl, Br, or I, preferably Cl or Br), for example, HCl or HBr in a polar protic solvent, for example, an alcohol (R₂OH), for example, MeOH, EtOH, n-butanol, isopropanol, or mixtures thereof. In some embodiments, HX gas is introduced directly into a solution of the benzonitrile of Formula 2.1 in R₂OH via a sparge tube. The reaction is conducted at a temperature from about −10° C. to about −5° C. and preferably from about 0° C. to about −5° C. during the HX sparge. It is preferred if the temperature is raised to about 25° C. following the addition of the HX. HX gas may be introduced into the reaction system at pressures ranging from about atmospheric pressure to about 3500 kPa. Alternatively, solutions of benzonitriles of Formula 2.1, in a variety of organic solvents, for example, tetrahydrofuran (THF), ethyl acetate (EtOAc), dichloromethane (CH₂Cl₂), toluene, or mixtures thereof, are treated with an anhydrous inorganic acid (HX), for example, HCl or HBr, in the presence of an alcohol (R₂OH). Molar ratios of benzonitriles of Formula 2.1 to the alcohol are from about 1:1 to about 1:10, however, molar ratios of about 1:1000 to about 1000:1 may also be used. In another embodiment, HX is generated in situ via the decomposition of an acyl halide, such as, for example, acetyl chloride and acetyl bromide, when said acyl halide is contacted with R₂OH. In another embodiment thionyl chloride is used as a source of HCl. In this method the acyl halide may be added to a solution of the benzonitrile of Formula 2.1 in R₂OH or may be added to the R₂OH first, followed by the addition of the benzonitrile of Formula 2.1 to the pre-formed solution of HX. In both cases, the reaction is conducted at a temperature from about −10° C. to about −5° C. and preferably from about 0° C. to about −5° C. during the HX formation, and preferably the temperature is raised to about 25° C. following the addition.

In some embodiments, subjecting benzonitriles of Formula 2.1, wherein R₁ is nitro or a carboxylate ester, to one of the described methods affords alkoxyimidate salts of Formula 2.2, wherein R₁ is as defined and R₂ is derived from R₂OH. In another embodiment, subjecting benzonitriles of Formula 2.1, wherein R₁ is a carboxylic acid, to one of the described methods affords alkoxyimidate salts of Formula 2.2, wherein R₁ is a mixture of the carboxylic acid and ester, wherein the R₁ ester and R₂ are both derived from R₂OH, e.g., when R₂OH is MeOH, R₁ is the methyl ester and R₂ is methyl.

[chemical structure]

1,3-Diaryltriazoles of Formula 3.2 can be prepared as illustrated in Scheme 3. In step a, haloalkoxyarylhydrazines of Formula 1.2 is reacted with an arylalkoxyimidate of Formula 2.2 to produce an intermediate iminohydrazine of Formula 3.1. In step b, the iminohydrazine is cyclized with a formate source, such as, for example, formic acid, formate esters, e.g., methyl- and ethyl formate, and orthoesters, e.g., trimethyl- and triethyl orthoformate, to afford said 1,3-diaryltriazoles of Formula 3.2.

In step a, solutions of arylalkoxyimidate salts of Formula 2.2 in a pyridine solvent, e.g., pyridine, 2-methylpyridine, 3-methylpyridine, 4-methylpyridine or lutidine, are reacted with haloalkoxyarylhydrazine salts ((HX)ₙ where n=0, 1, or 2) of Formula 1.2 to produce an intermediate iminohydrazine of Formula 3.1 Approximately, a 1:1 molar ratio of the haloalkoxyarylhydrazine salts of Formula 1.2 and the arylalkoxyimidate salts of Formula 2.2 may be used, however, molar ratios of about 5:1 to about 1:5 may also be used. The reaction is conducted at a temperature from about −10° C. to about 10° C. and preferably from about 0° C. to about −5° C. during the addition of the hydrazine, and then the temperature is preferably raised to about 25° C. following the addition.

In step b, the intermediate iminohydrazine of Formula 3.1 is cyclized using a formate source. The reaction is conducted at a temperature from about 20° C. to about 100° C. and preferably from about 95° C. to about 100° C., to form the 1,3-diaryltriazole of Formula 3.2.

Iminohydrazines of Formula 3.1 that are of particular usefulness in producing (substituted-phenyl)-triazolyl-(substituted-phenyl) molecules that are of particular interest are:

[chemical structure]

[chemical structure]

1,3-Diaryl triazole compounds of Formula 4.2 and Formula 4.3, can be prepared according to Scheme 4. In method a, intermediate 1,3-diaryltriazoles of Formula 3.2, wherein R₁ is an ester, can be saponified to give 1,3-diaryltriazoles substituted with a carboxylic acid of Formula 4.2. In method b, intermediate 1,3-diaryltriazole of Formula 3.2), wherein R₁ is nitro, can be reduced to give 1,3-diaryltriazoles substituted with an amine of Formula 4.3.

Method a can be conducted in a polar protic solvent, such as an alcohol, for example, MeOH, EtOH, n-butanol, isopropanol, and mixtures thereof, or in a polar, aprotic solvent such as THF, in the presence of an alkali hydroxide base, such as, for example, sodium (NaOH), potassium (KOH), or lithium hydroxide (LiOH), and water. The reaction can be conducted at a temperature from about 20° C. to about 60° C. and preferably from about 20° C. to about 30° C. The pH of the reaction mixture is from about 8 to about 14 and preferably from about 10 to about 12.

Method b can be carried out in a wide variety of organic solvents including, for example, polar protic solvents, such as alcohols, e.g., MeOH, EtOH, n-butanol, isopropanol, and mixtures thereof, polar aprotic solvents, such as THF and EtOAc, or organic acids, such as, for example, AcOH, in the presence of a catalyst, such as palladium on carbon, and a hydrogen source, such as, for example hydrogen gas, ammonium salts, e.g., ammonium formate, and cyclohexadiene. The reaction can be conducted at a temperature from about 20° C. to about 50° C. and preferably from about 20° C. to about 30° C. The reaction can be conducted at a pressure from about 100 kPa to about 700 kPa and preferably from about 100 kPa to about 350 kPa.

1,3-Diaryltriazole of Formula 4.2 and Formula 4.3 can be used as intermediates to form pesticides disclosed in U.S. Pat. No. 8,178,658 as disclosed therein.

### EXAMPLES

These examples are for illustration purposes and are not to be construed as limiting the disclosure to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Examples for which the temperature is described as “room temperature” were conducted in climate controlled laboratories with temperatures ranging from about 20° C. to about 24° C. Molecules are given their knownnames, named according to naming programs within ISIS Draw, Chem Draw or ACD Name Pro. If such programs are unable to name a molecule, the molecule is named using conventional naming rules. ¹H NMR spectral data are in ppm (δ) and were recorded at 300, 400 or 600 MHz, and ¹³C NMR spectral data are in ppm (δ) and were recorded at 75, 100 or 150 MHz, unless otherwise stated.

### Example 1

### Preparation of (4-(perfluoroethoxy)phenyl)hydrazine

[chemical structure]

To a dry 500 mL round bottomed flask equipped with magnetic stirrer, nitrogen inlet, addition funnel, and thermometer, were charged 4-perfluoroethoxyaniline (11.8 g, 52.0 mmol), HCl (2 N, 100 mL), and the resulting suspension was cooled to about 0° C. with an external ice/salt (sodium chloride, NaCl) bath. To the suspension was added a solution of NaNO₂ (1.05 g, 54.5 mmol) in water (10 mL) dropwise from the addition funnel at a rate which maintained the temperature below 5° C., and the resulting colorless solution was stirred at 0° C. for 30 minutes (min). To a separate 500 mL round bottomed flask equipped with magnetic stir bar, addition funnel, and thermometer were added Na₂S₂O₄ (27.1 g, 156 mmol), NaOH (1.04 g, 26.0 mmol), and water (60 mL), and the suspension was cooled to about 5° C. with an external cooling bath. The diazonium salt solution prepared in round bottom 1 was transferred to the addition funnel and added to round bottom 2, containing the Na₂S₂O₄—NaOH suspension, at a rate which maintained the temperature below 8° C. Following the addition, the reaction mixture was warmed to 18° C. and the pH was adjusted to about 8 with NaOH (50%). The resulting pale orange solution was extracted with EtOAc (3×100 mL) and the combined organic extracts were washed with water (100 mL), washed with saturated aqueous NaCl solution (100 mL), dried over anhydrous magnesium sulfate (MgSO₄), filtered, and the filtrate concentrated to give the crude product as an orange semi-solid (12.2 g). The orange semi-solid was purified by flash column chromatography using 0-100% EtOAc/hexanes as eluent to give the title compound as a yellow liquid (10.4 g, 83%): ¹H NMR (400 MHz, CDCl₃) δ 7.18-7.00 (m, 2H), 6.97-6.68 (m, 2H), 5.24 (bs, 1H), 3.98-3.09 (bs, 2H); ¹⁹F NMR (376 MHz, CDCl₃) δ −86.00, −86.01, −87.92; EIMS m/z 242 ([M]⁺).

### Example 2

### Preparation of methyl 4-(1-(4-(trifluoromethoxy)phenyl)-1H-1,2,4-triazol-3-yl)benzoate

[chemical structure]

To a magnetically stirred solution of methyl 4-(imino(methoxy)methyl)benzoate hydrochloride (1.15 g, 5.00 mmol) in anhydrous pyridine (5 mL) cooled by an ice bath was added (4-(trifluoromethoxy)phenyl)hydrazine hydrochloride (1.14 g, 5.00 mmol) in several portions. After warming to room temperature (RT) and stirring overnight (18 hours (h)), the yellow reaction mixture was diluted with water (25 mL) and washed with CH₂Cl₂ (2×50 mL). The combined organic layers were washed with brine, dried (MgSO₄) and concentrated to give an orange yellow solid (1.60 g). The orange yellow solid was dissolved in formic acid (15 mL) and heated at reflux for 8 h. The reaction mixture was cooled to RT, diluted with water and washed with ether (2×50 mL). The ether layers were washed with water (3×50 mL), brine (50 mL) and dried over MgSO₄. Concentration furnished the title compound as a tan solid (1.42 g, 78%). A small sample was purified by flash column chromatography using 0-100% EtOAc/hexanes as eluent to give an off-white solid: mp 171-172° C.; ¹H NMR (400 MHz, CDCl₃) δ 8.60 (s, 1H), 8.27 (m, 2H), 8.15 (m, 2H), 7.81 (m, 2H), 7.40 (d, J=8.5 Hz, 2H), 3.95 (s, 3H); ¹³C NMR (101 MHz, CDCl₃) δ 166.76, 162.61, 148.55, 141.77, 135.41, 134.46, 131.03, 130.02, 126.46, 122.44, 121.66, 121.31, 119.10; EIMS m/z 363 ([M]⁺).

### Example 3

### Preparation of 4-(1-(4-(perfluoroethoxy)phenyl)-1H-1,2,4-triazol-3-yl)benzoic acid

[chemical structure]

A 1 L, three-neck round bottom flask, fitted with mechanical stirring, temperature probe and nitrogen inlet, was charged with 4-(ethoxy(imino)methyl)benzoic acid hydrochloride (25 g, 109 mmol) and pyridine (200 mL). The white suspension was cooled to 5° C. and (4-(perfluoroethoxy)phenyl)hydrazine hydrochloride (30.9 g, 109 mmol) was added in portions. The white suspension turned yellow and the temperature rose to 5.7° C. The cold bath was removed and the reaction mixture was allowed to warm slowly. At about 12° C., the contents of the flask thickened to where stirring was not useful. Additional pyridine (20 mL) was added to try to make it more fluid. After 20 min the reaction mixture was poured into water (400 mL) and a flocculent solid crashed out. The mixture was extracted with CH₂Cl₂ (1×400 mL), the phases separated, and the solid on top of the aqueous layer collected by vacuum filtration. The aqueous filtrate was extracted with CH₂Cl₂ (2×200 mL) and the organic extracts were combined. The solid was washed twice with CH₂Cl₂ and the organic washes were combined with the original organic extracts. The combined organic extracts were washed with water (2×500 mL) and the water washes were back-extracted with CH₂Cl₂ until the aqueous layer was colorless (1×200 mL). The organic extracts were concentrated under reduced pressure to yield a dark-red oil (17.2 g). The solid that was isolated earlier turned light-salmon upon standing in air. This was suspended in CH₂Cl₂ (500 mL), stirred for 5 min, filtered, rinsed with CH₂Cl₂ and dried at 50° C. in a vacuum oven to yield a bright yellow solid (20 g).

A 500 mL, three-necked, round bottom flask, fitted with magnetic stirring, temperature probe and nitrogen inlet, was charged with the dark red oil (17.2 g), the bright yellow solid (20 g) and formic acid (200 mL). The mixture was heated at 100° C. for 16 h. The heat was removed and the mixture was allowed to cool. At 90° C., a light-yellow solid crashed out. The mixture was cooled to 23° C. and water (200 mL) was added. The mixture was stirred for 1 hour and the solid collected by vacuum filtration, washed with water and air dried. The solid was placed in a vacuum oven at 50° C. for 2 days to furnish the title compound as a light tan solid (22.3 g, 51%): ¹H NMR (400 MHz, DMSO-d₆) δ 13.13 (s, 1H), 9.48 (s, 1H), 8.23 (m, 2H), 8.10 (m, 4H), 7.64 (m, 2H); ESIMS m/z 400 ([M+H]⁺).

### Example 4

### Preparation of 3-(4-nitrophenyl)-1-(4-(perfluoroethoxy)phenyl)-1H-1,2,4-triazole

[chemical structure]

To a stirred solution of ethyl 4-nitrobenzimidate hydrochloride (3 g, 13 mmol) in pyridine (13 mL) at 0° C. was added (4-(perfluoroethoxy)phenyl)hydrazine hydrochloride (3.62 g, 13.0 mmol) in three portions. The reaction mixture was warmed to RT for 2 h and was diluted with water and CH₂Cl₂. The aqueous layer was washed with CH₂Cl₂ (3×20 mL), and the combined organic fractions were washed with water (30 mL) and dried over MgSO₄. Concentration gave a sticky red solid: ¹H NMR (400 MHz, CDCl₃) δ 8.34-8.23 (m, 2H), 8.00-7.90 (m, 2H), 7.20-7.07 (m, 4H), 6.33 (s 1H), 4.66 (s, 2H); ESIMS m/z 390 ([M]⁺).

The sticky red solid was added to formic acid (30 mL) and heated at 100° C. for 18 h. The reaction mixture was cooled to RT and added to cold water. The precipitate that formed was collected on a fritted glass funnel, washed with water, air dried and placed under vacuum to give the title compound as a light pink solid (4.99 g, 96%): mp 132-135° C.; ¹H NMR (400 MHz, CDCl₃) δ 8.68 (s, 1H), 8.43-8.31 (m, 4H), 7.89-7.80 (m, 2H), 7.49-7.38 (m, 2H); ESIMS m/z 400 ([M]+).

### Example 5

### Preparation of 3-(4-nitrophenyl)-1-(4-(trifluoromethoxy)phenyl)-1H-1,2,4-triazole

[chemical structure]

Methyl 4-nitrobenzimidate hydrochloride (0.53 g, 2.4 mmol) was dissolved in pyridine (2.5 mL) and cooled to 0° C. To this stirred solution was added (4-(trifluoromethoxy)phenyl)hydrazine hydrochloride (0.559 g, 2.45 mmol) in three portions, and the dark red solution was warmed to RT for 18 h. The reaction mixture was then diluted with water and CH₂Cl₂. The aqueous layer was washed with CH₂Cl₂, and the combined organic layers were washed with a saturated solution of NH₄Cl and dried over MgSO₄. Concentration followed by purification by flash column chromatography using 0-70% EtOAc/hexanes as eluent gave 4-nitro-N′-(4-(trifluoromethoxy)phenyl)benzimidohydrazide as a red solid (0.47 g, 57%): ¹H NMR (400 MHz, CDCl₃) δ 8.31-8.23 (m, 2H), 7.99-7.90 (m, 2H), 7.20-7.06 (m, 4H), 6.47 (s, 1H), 4.71 (s, 2H); ¹³C NMR (101 MHz, CDCl₃) δ 148.24, 145.70, 145.55, 142.84, 139.89, 126.26, 123.85, 122.24, 121.92, 119.37, 114.82; ESIMS m/z 340 ([M+H]⁺).

The 4-nitro-N′-(4-(trifluoromethoxy)phenyl)benzimidohydrazide (0.47 g, 1.38 mmol) was taken up in formic acid (20 mL) and refluxed for 18 h at 100° C. The reaction was diluted with water and a solid precipitated from solution. The solid was collected on a fritted glass funnel, washed with water and air dried to give the title compound as a pink solid (0.655 g, 95%): ¹H NMR (400 MHz, CDCl₃) δ 8.63 (s, 1H), 8.45-8.27 (m, 4H), 7.93-7.73 (m, 2H), 7.42 (d, J=8.5 Hz, 2H); ¹³C NMR (101 MHz, CDCl₃) δ 172.76, 153.40, 148.53, 142.08, 136.30, 135.23, 127.31, 124.06, 123.95, 122.50, 121.40, 77.22; ESIMS m/z 350 ([M]⁺).

### Example 6

### Preparation of 4-(1-(4-(trifluoromethoxy)phenyl)-1H-1,2,4-triazol-3-yl)benzoic acid

[chemical structure]

To methyl 4-(1-(4-(trifluoromethoxy)phenyl)-1H-1,2,4-triazol-3-yl)benzoate (0.332 g, 0.914 mmol) in THF (6 mL) and water (3 mL) was added lithium hydroxide (0.066 g, 2.74 mmol). The solution immediately turned from yellow to orange-red. The reaction was stirred vigorously at RT for 16 h. The solution was acidified to pH 2 and diluted with water and CH₂Cl₂. The layers were separated and the aqueous layer was extracted with EtOAc (3×10 mL) and the combined organic fractions were washed with water (10 mL) and brine (10 mL), dried over MgSO₄, filtered and concentrated to give the title compound as a tan solid (0.29 g, 91%): mp 228-233° C.; ¹H NMR (400 MHz, DMSO-d₆) δ 10.55-10.24 (m, 1H), 9.46 (s, 1H), 8.23 (d, J=8.0 Hz, 2H), 8.09 (d, J=7.9 Hz, 4H), 7.64 (d, J=8.5 Hz, 2H); ESIMS m/z 350 ([M+H]⁺).

### Example 7

### Preparation of 4-(1-(4-(perfluoroethoxy)phenyl)-1H-1,2,4-triazol-3-yl)benzoic acid

[chemical structure]

In a 250 mL round bottomed flask equipped with an overhead stirrer, T-type thermocouple, and nitrogen inlet was added methyl 4-(1-(4-(perfluoroethoxy)phenyl)-1H-1,2,4-triazol-3-yl)benzoate (11.1 g, 26.9 mmol) and THF (100 mL). To this yellow suspension was added water (10 mL) and lithium hydroxide monohydrate (3.4 g, 81 mmol). There was no change in reaction appearance and temperature (20.5° C.). The reaction was stirred at 23° C. for 39 h during which it became a yellow solution. The reaction was analyzed by LCMS which showed that the reaction had progressed only 23%. A heating mantle was attached to the reaction flask and the flask was heated to an internal temperature of 60° C. Analysis by LCMS showed the reaction to be complete. The reaction was cooled to 4° C. in an icebath and water (100 mL) was added providing a light yellow solution. Concentrated HCl (8.0 g) was added (note: exothermic) which gave a thick white precipitate. The white suspension was stirred at 5° C. for 30 min then the solid was collected by vacuum filtration and washed with water (2×25 mL). The white wet cake was allowed to dry in air for 3 h, and then placed into a vacuum oven (50° C., 700 mm Hg vacuum 16 h). This gave the title compound as a white solid (10.3 g, 96%): mp 227-229° C.; ¹H NMR (400 MHz, CDCl₃) δ 8.65 (s, 1H), 8.32 (d, J=8.4 Hz, 2H), 8.23 (d, J=8.4 Hz, 2H), 7.84 (d, J=8.9 Hz, 1H), 7.42 (d, J=8.9 Hz, 2H).

### Example 8

### Preparation of (4-(perfluoroethoxy)phenyl)hydrazine hydrochloride

[chemical structure]

Step 1. Preparation of 1-(diphenylmethylene)-2-(4-(perfluoroethoxy)phenyl)-hydrazine: To a dry 2 L round bottomed flask fitted with an overhead mechanical stirrer, nitrogen inlet, thermometer, and reflux condenser were added 1 bromo-4-(perfluoroethoxy)-benzene (100 g, 344 mmol), benzophenone hydrazone (74.2 g, 378 mmol), and (2,2′-bis(diphenylphosphino)-1,1′-binaphthyl) (BINAP, 4.28 g, 6.87 mmol), and the mixture was suspended in anhydrous toluene (500 mL). To exclude oxygen, argon was sparged into the mixture for ten minutes (min) prior to and during the addition of palladium (II) acetate (Pd(OAc)₂, 1.54 g, 6.87 mmol) and sodium tert-butoxide (NaO^{t}Bu, 49.5 g, 515 mmol), which was added in portions. The argon sparge was halted and the brown mixture was warmed to 100° C. and stirred for 3 h. The reaction was cooled to RT and poured into water (500 mL) and the aqueous mixture was extracted with EtOAc (3×200 mL). The combined organic extracts were washed with water, washed with saturated aqueous NaCl, dried over anhydrous MgSO₄, filtered, and concentrated under reduced pressure on a rotary evaporator. The crude product was purified by flash column chromatography using 0-100% (v/v) EtOAc/hexanes as eluent to give the title compound as a red oil (123.3 g, 88%): ¹H NMR (400 MHz, CDCl₃) δ δ 7.63-7.56 (m, 4H), 7.55 (t, J=1.5 Hz, 1H), 7.51 (d, J=4.7 Hz, 1H), 7.36-7.26 (m, 5H), 7.13-7.04 (m, 4H); ¹⁹F NMR (376 MHz, CDCl₃) 6-85.94, −87.84; ¹³C NMR (101 MHz, CDCl₃) δ 145.23, 143.46, 141.24, 138.06, 132.53, 129.74, 129.41, 129.03, 128.30, 128.23, 126.57, 122.82, 113.45.

Step 2. Preparation of (4-(perfluoroethoxy)phenyl)hydrazine hydrochloride: To a dry 250 mL round bottomed flask equipped with a magnetic stir bar, thermometer, and reflux condenser were added 1-(diphenylmethylene)-2-(4-(perfluoroethoxy)phenyl)hydrazine (63.6 g, 157 mmol), EtOH (50 mL), and concentrated HCl (100 mL, about 1.20 mol), and the reaction was warmed to 85° C. and stirred for 5 h. The reaction was cooled to RT and the dark slurry was concentrated to a brown paste on a rotary evaporator. The paste was slurried in CH₂Cl₂ (200 mL) and the resulting solid was collected by vacuum filtration and dried under vacuum at 40° C. to give the title compound as a tan solid (36.0 g, 82%): ¹H NMR (400 MHz, DMSO-d₆) δ 10.47 (s, 3H), 8.62 (s, 1H), 7.43-7.18 (m, 2H), 7.20-6.93 (m, 2H); ¹⁹F NMR (376 MHz, DMSO-d₆) δ −85.30, −87.02; ESIMS m/z 243.15 ([M+H]⁺).

### Example 9

### Preparation of methyl 4-(imino(methoxy)methyl)benzoate hydrochloride

[chemical structure]

To a magnetically stirred solution of methyl 4-cyanobenzoate (12.5 g, 78 mmol) in benzene (25 mL) and MeOH (7 mL) cooled to 0° C. was bubbled anhydrous HCl subsurface for 3 h. After storing in the refrigerator overnight, a heavy white precipitate formed. The solid was filtered through a fritted glass funnel and washed with diethyl ether to furnish the title compound as a white solid (17.5 g, 96%): mp 209-210° C.; ¹H NMR (400 MHz, CDCl₃) δ 13.05 (br s, 1H), 12.32 (br s, 1H), 8.48 (m, 2H), 8.22 (m, 2H), 4.60 (s, 3H), 3.97 (s, 3H); ¹³C NMR (101 MHz, DMSO-d₆) δ 166.98, 165.69, 138.38, 131.74, 129.00, 127.78, 52.29, 26.16; EIMS m/z 192 ([M]⁺).

### Example 10

### Preparation of methyl 4-(ethoxy(imino)methyl)benzoate hydrochloride

[chemical structure]

A 2 L, three-necked round bottomed flask equipped with a magnetic stir bar, a temperature probe, addition funnel and nitrogen inlet was charged with methyl 4-cyanobenzoate (100 g, 620 mmol). The methyl 4-cyanobenzoate was dissolved in EtOH (438 mL) and cooled in an ice bath to 0° C. Acetyl chloride (353 mL, 4960 mmol) was added dropwise into the stirring solution over a 2 h period during which time an exotherm from 0° C. to 21° C. was noted. The reaction flask was capped, sealed with Parafilm®, and allowed to stir at 23° C. for 18 h. The resulting white solid was collected by vacuum filtration and washed with EtOH. The filtrate was concentrated until it became turbid and was then cooled in an ice bath. The resulting precipitate was collected by vacuum filtration, rinsed with EtOH, and the filtrate treated as described to give another crop. The solids were dried to give the title compound as a white solid (128 g, 85%): ¹H NMR (400 MHz, CDCl₃) δ 12.85 (br s, 1H), 12.20 (br s, 1H), 8.49 (m, 2H), 8.23 (m, 2H), 5.00 (q, 2H), 4.00 (s, 3H), 1.72 (t, 3H).

### Example 11

### Preparation of 4-(ethoxy(imino)methyl)benzoic acid hydrochloride and ethyl 4-(ethoxy(imino)methyl)benzoate hydrochloride

[chemical structure]

A 500 mL, three-necked flask, equipped with a magnetic stir bar, nitrogen inlet, addition funnel, and a temperature probe was charged with anhydrous EtOH (125 mL). The vessel was cooled to 5° C. and acetyl chloride (97 mL, 1332 mmol) was added at a rate that maintained the temperature range of 5° C. to 10° C. When the addition was complete, 4-cyanobenzoic acid (25 g, 167 mmol) was added in portions over 15 min. No exotherm was noted during the addition of the solid. When the addition was complete, the white suspension was allowed to warm to 25° C. The reaction vessel was sealed with Parafilm® and stirred at 23° C. for 18 h. The white suspension was vacuum filtered and the solid was rinsed with EtOH and dried to constant mass, furnishing 4-(ethoxy(imino)methyl)benzoic acid hydrochloride as a white solid (25 g, 65%): ¹H NMR (400 MHz, DMSO-d₆) δ 12.44 (br s, 1H), 8.26 (m, 2H), 8.14 (m, 2H), 4.70 (q, 2H), 1.51 (t, 3H). The filtrate was concentrated and treated with ether to give a white solid. The solid was collected by vacuum filtration and rinsed with ether to give ethyl 4-(ethoxy(imino)methyl)benzoate hydrochloride as a white solid (11 g, 25%): ¹H NMR (400 MHz, DMSO-d₆) δ 12.07 (br s, 1H), 8.24 (m, 2H), 8.15 (m, 2H), 4.66 (q, 3H), 4.37 (q, 3H), 1.49 (t, 3H), 1.35 (q, 3H).

### Example 12

### Preparation of ethyl 4-nitrobenzimidate hydrochloride

[chemical structure]

To a solution of 4-nitrobenzonitrile (27 g, 182 mmol) in EtOH (128 ml, 2187 mmol) under nitrogen was added acetyl chloride (104 ml, 1458 mmol) dropwise at 0° C. over 1 h, and the reaction was warmed to RT. The flask was sealed and the reaction was stirred for 56 h. The resulting precipitate (4-nitrobenzamide) was collected by filtration, and the filtrate was treated with diethyl ether. The resulting precipitate was collected by filtration, washed with diethyl ether and air dried to give the title compound (26.7 g, 58%): mp 198-200° C.; ¹H NMR (400 MHz, DMSO-d₆) δ 8.37-8.30 (m, 2H), 8.21-8.13 (m, 2H), 7.35 (s, 1H), 7.22 (s, 1H), 7.09 (s, 1H), 4.34 (q, J=7.1 Hz, 2H), 1.32 (t, J=7.1 Hz, 3H); EIMS m/z 193 [M]⁺.

What is claimed is:

1. A process comprising: [chemical structure]
(1a) reacting a haloalkoxyarylhydrazine of Formula 1.2 with an arylalkoxyimidate of Formula 2.2 to produce an intermediate iminohydrazine of Formula 3.1.; followed by
(1b) cyclizing said iminohydrazine of Formula 3.1 with a formate source to produce the 1,3-diaryltriazole of Formula 3.2;
wherein R is (C₁-C₆)haloalkoxy, R₁ is NO₂, C(═O)OH, or C(═O)O(C₁-C₄)alkyl, and R₂ is a (C₁-C₆)alkyl.

2. A process according to claim 1 wherein said formate source is formic acid, methyl formate, ethyl formate, trimethyl orthoformate, triethyl orthoformate, or mixtures thereof.

3. A process according to claim 1 wherein said formate source is formic acid.

4. A process according to claim 1 wherein said reaction (1a) is conducted in a solvent selected from pyridine, 2-methylpyridine, 3-methylpyridine, 4-methylpyridine, lutidine, or mixtures thereof.

5. A process according to claim 1 wherein the molar ratio of said haloalkoxyarylhydrazine salts of Formula 1.2 to said arylalkoxyimidate salts of Formula 2.2 is from about 5:1 to about 1:5.

6. A process according to claim 1 wherein the reaction (1a) is conducted at a temperature from about −10 to about 10° C.

7. A process according to claim 1 wherein said cyclizing reaction (1b) is conducted at a temperature from about 20 to about 100° C.

8. Iminohydrazines of Formula 3.1 having the following structures [chemical structure]
